(19) United States
(12) Patent Application Publication
(10) Pub. No.: US 2010/0106059 A1
(43) Pub. Date: Apr. 29, 2010

(54) SYSTEM AND METHOD FOR EVALUATING NEUROMUSCULAR AND JOINT PROPERTIES

(75) Inventors: Li-Qun Zhang, Wilmette, IL (US); Hyung-Soon Park, Rockville, MD (US); Yupeng Ren, Chicago, IL (US)

Correspondence Address: HUSCH BLACKWELL SANDERS LLP, 190 Carondelet Plaza, Suite 600, ST. LOUIS, MO, 63105, US

(73) Assignee: REHABTEK LLC, Wilmette, IL (US)

(21) Appl. No.: 12/532,626

(22) PCT Filed: Mar. 24, 2008
(86) PCT No.: PCT/US2008/058076

§ 371 Date: Nov. 3, 2009

Related U.S. Application Data
(60) Provisional application No. 60/919,402, filed on Mar. 22, 2007.

(51) Int. Cl. A61B 5/103

(52) U.S. Cl. 600/587

(57) ABSTRACT

A pocket neuromuscular evaluator delivers controlled tendon taps, makes quantitative measures of the taps and the reflex responses invoked, evaluates not only the neurological reflexes but also the muscle-joint properties, analyzes the data, displays the results, and records them to provide quantitative characterizations of the neuromuscular and joint properties.

### CROSS-REFERENCE TO RELATED APPLICATIONS

This application claims priority from U.S. Provisional Patent Application No. 60/919,402, filed on Mar. 22, 2007.

### FIELD OF INVENTION

The present invention relates to the field of medical diagnosis and rehabilitation using a portable device for diagnosing and evaluating neurological reflex properties and biomechanical properties of the muscles and joints. More specifically, a device delivers controlled tendon taps to induce tendon reflex, and measures various neuromuscular and biomechanical properties including reflex gains, reflex threshold, natural undamped frequency and damping ratio of the muscle-joint system, stiffness, viscous damping, joint range of motion, muscle strength, and voluntary control ability.

### BACKGROUND OF INVENTION

Tendon reflexes have been very widely used to evaluate the nervous system. Almost every clinician uses a tendon reflex hammer to obtain a quick evaluation of tendon reflexes in clinical practice. They may check several common tendon sites in the upper and lower limbs and they may tap several times at each tendon to obtain a quick and convenient evaluation of the nervous system. However, tendon reflexes elicited with a traditional reflex hammer vary substantially and are dependent on how accurate and how strong the tendon is tapped and the response is graded subjectively by the clinician on a five-point scale ranging from 0 to 4, with 0 being no reflex response, 1 for low average, 2 for average normal, 3 for brisk than average, and 4 for hyperactive and association with clonus (Bates (1991) A guide to physical examination and history taking).

Taken in conjunction with other measures on the neuromuscular system, eliciting tendon reflexes is very useful in quick examination of nervous system. However, the measurements are qualitative and subjective in nature, with limited inter-rater reliability (Marshall and Little (2002) J Spinal Cord Med 25: 94-99). As pointed in a recent review, deep tendon reflexes are extremely variable and may be misleading if used on their own (Dick (2003) J Neurol Neurosurg Psych 74: 150-153). There is a particular need to quantify tendon reflexes more accurately.

One problem with the traditional manual tendon tapping is the variation in the spot at which the tendon hammer hits the tendon. When a clinician swings the reflex hammer to hit the tendon, he or she often hits different spots from tap to tap. Such variations may cause considerable variation in the reflex responses. Several studies have been done to use motorized hammers to tap the tendons for more accurate control of the tapping. However, the devices are not portable and were only be used in research labs. The pocket neuromuscular evaluator addresses the problem by finding the most sensitive spot and tapping consistently with quantitative measures.

Another aspect in evaluating tendon reflexes is the reflex threshold, the stimulus threshold for eliciting reflex responses. For example, spastic hypertonia of neurologically impaired patients is associated with reduction in the reflex threshold, and reflex hyperexcitability in neurological impairments is also reflected in the higher sensitivity to a stimulus (Powers et al. (1988) Ann Neurol 23: 115-124; Rymer and Katz (1994) Phys Med Rehab 8: 441-454; Zhang et al. (2000) Arch Phys Med Rehab 81: 901-909). It is not clear whether hyperactive reflexes in neurological impairment are due to an increase in reflex gain (Ibrahim et al. (1993) Brain 116: 971-989; Rack et al. (1984) Brain 107: 637-654; Thilmann et al. (1991) Brain 114: 233-244; Zhang et al. (2000) Arch Phys Med Rehab 81: 901-909) or a decrease in reflex threshold (Powers et al. (1988) Annals of Neurology 23: 115-124; Zhang et al. (2000) Arch Phys Med Rehab 81: 901-909). In clinical practice, a clinician usually taps the tendons of patients with spastic hypertonia with lighter taps as compared to tapping of health subjects. However, there is no quantitative evaluation on the changes in reflex threshold, mainly due to the difficulty in obtaining such a measure. The neuromuscular evaluator measures not only the reflex responses (reflex gain) but also reflex threshold in the tapping force as another measure of excitability of the human reflex system. Furthermore, it relates the reflex-mediated response (limb movement) to the tapping force, treats them as the system output and input, respectively, and evaluates the reflex system properties.

Considering that the tendon reflexes can be extremely variable and may be misleading if used on their own (Dick (2003) J Neurol Neurosurg Psychiatry 74: 150-153), reflex examinations should be done in combination with other related components such as muscle and joint properties to be more reliable and accurate. The neuromuscular evaluator provides quantitative and convenient measures of both reflex excitability (reflex gain and threshold) and muscle-joint biomechanical properties (natural undamped frequency and damping ratio, which are related to joint stiffness and viscous damping), based on previous work on evaluations of reflex (Chung et al. (2005) Arch Phys Med Rehab 86: 318-327; Zhang et al. (1999) IEEE Trans Rehab Eng 7: 193-203; Zhang et al. (2000) Arch Phys Med Rehab 81: 901-909) and nonreflex (Chung et al. (2004) Arch Phys Med Rehab 85: 1638-1646) changes.

### SUMMARY OF INVENTION

The present invention describes methods and apparatus to deliver tendon taps under precise control, measure the tapping force and the reflex responses invoked, evaluate not only the neurological reflexes but also the related muscle-joint properties, analyze the data in real-time, display the results, and record them to provide quantitative characterizations of the neuromuscular and biomechanical properties. Practically, the neuromuscular evaluator is user-friendly and pocket sized, making it suitable for quantitative evaluations of both neurological reflexes and muscle-joint properties and their changes associated with neurological impairments and musculoskeletal diseases in a clinical setting.

One part of the pocket evaluator is a motorized tapping mechanism composed of an actuating mechanism that generates linear motion, force sensor, and central processing unit that controls the tap, analyzes and displays the data. The actuating mechanism includes impact generator such as an electric rotary motor and rotary-to-linear motion converter. The central processing unit controls the speed and torque of the motor, analyzes the collected data, and displays the results. A small gyroscope sensor is attached on the limb, which measures the rate change of the joint angle in real-time.

The apparatus is capable of tapping with controlled force and measuring reflex responses quantitatively including reflex gains and reflex threshold. The apparatus also provides precise measurements of musculoskeletal properties such as the natural undamped frequency and damping ratio of the muscle-joint system, stiffness, viscous damping, joint range of motion, muscle strength, and voluntary control ability.

### BRIEF DESCRIPTION OF THE DRAWINGS

FIG. 1 describes the mechanical design of the pocket neuromuscular evaluator

FIG. 2 illustrates the mechanism converting rotary motion to linear motion

FIG. 3 describes the spring mechanism

FIG. 4 describes two different methods for mounting force sensor

FIG. 5 describes the neuromuscular evaluator without force sensor

FIG. 6 illustrates typical usage of the pocket neuromuscular evaluator

FIG. 7 illustrates the design of supporting posts

FIG. 8 presents the block diagram and the flowchart describing the control algorithm

FIG. 9˜11 present representative tendon tapping and evaluation results.

### DETAILED DESCRIPTION OF THE INVENTION

### 1. Design of the Pocket Neuromuscular Evaluator

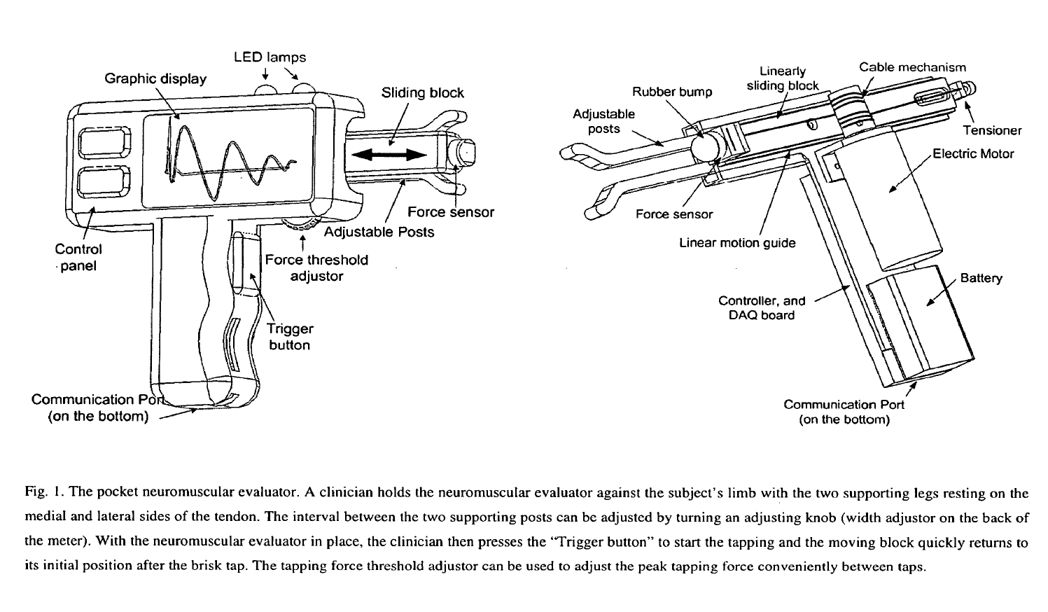

A pocket neuromuscular evaluator is designed to evaluate tendon reflexes conveniently at multiple tendon sites, including the patellar and Achilles tendons of the lower limb and the triceps and biceps brachii tendons of the arm. The neuromuscular evaluator is small in size and a clinician can hold it in hand comfortably during the testing (FIG. 1). Yet it has the capabilities of precise tapping control, reflex response measurements, data acquisition and analysis, and result display, and record saving (FIG. 1).

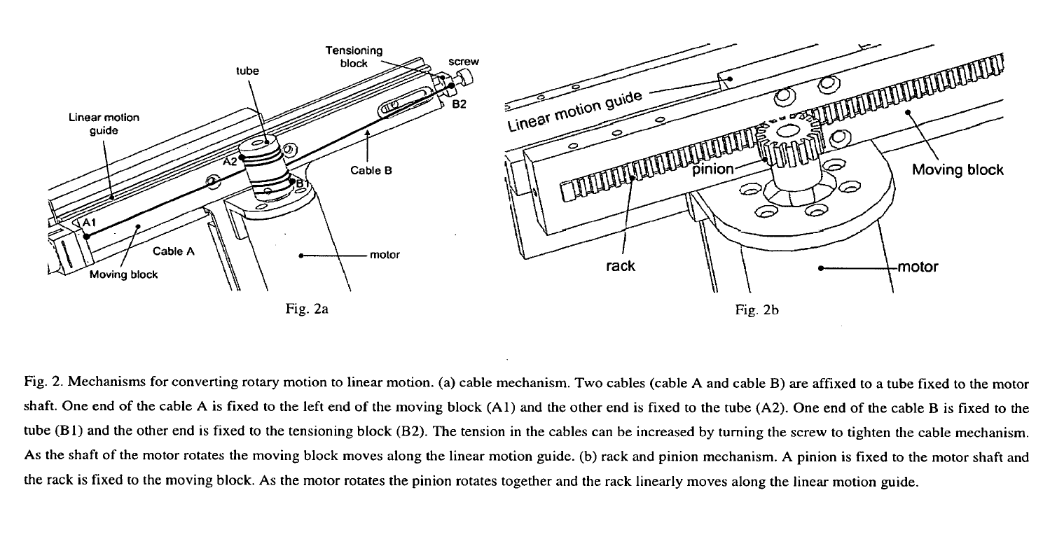

A small servomotor inside the neuromuscular evaluator delivers well-controlled taps onto the tendon repeatedly under the control of a microcontroller. A cable-driven mechanism or rack-and-pinion mechanism is used to convert the motor rotation into fast linear motion (FIG. 2). The cable-driven mechanism can produce very fast linear movement with lower friction, which is important in producing a brisk tap onto the tendon. Two cables (cable A and cable B) are affixed to a tube fixed to the motor shaft (FIG. 2a). One end of the cable A is fixed to the left end of the moving block (A1) and the other end is fixed to the tube (A2). One end of the cable B is fixed to the tube (B1) and the other end is fixed to the tensioning block (B2). The tension in the cables can be adjusted by turning the screw to tighten/loosen the cable mechanism. As the shaft of the motor rotates the moving block moves along the linear motion guide.

Alternatively, the rack-and-pinion mechanism can be used and implement by using off-shelf products. A pinion is fixed to the motor shaft and the rack is fixed to the moving block. As the motor rotates the pinion rotates together and the rack linearly moves along the linear motion guide (FIG. 2b). The rack-and-pinion mechanism coverts the motor rotation to fast linear motion, generating strong impact to the tendon using a small motor. The moving block is mounted on a linear motion guide so the motion is guided precisely and smoothly along the rail and the linear motion is controlled by a servomotor to tap the tendon in a well-controlled manner. A force sensor mounted on the front of the moving block or mounted next to the tendon measures the tapping force.

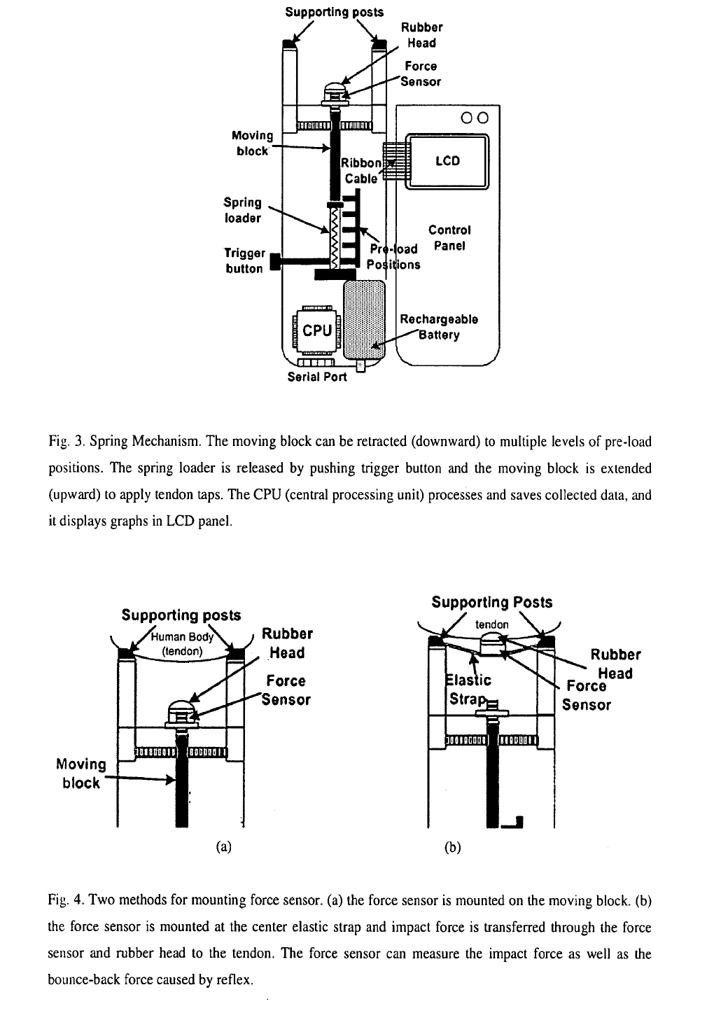

Actuation by spring mechanism is another alternative which does not need electric motor (FIG. 3). A rubber head and a force sensor are mounted on the moving block which is loaded to multiple pre-load positions. The apparatus can apply greater impact force as the moving block is loaded to lower positions. The use pushes trigger button to tap the tendon after placing the supporting posts properly.

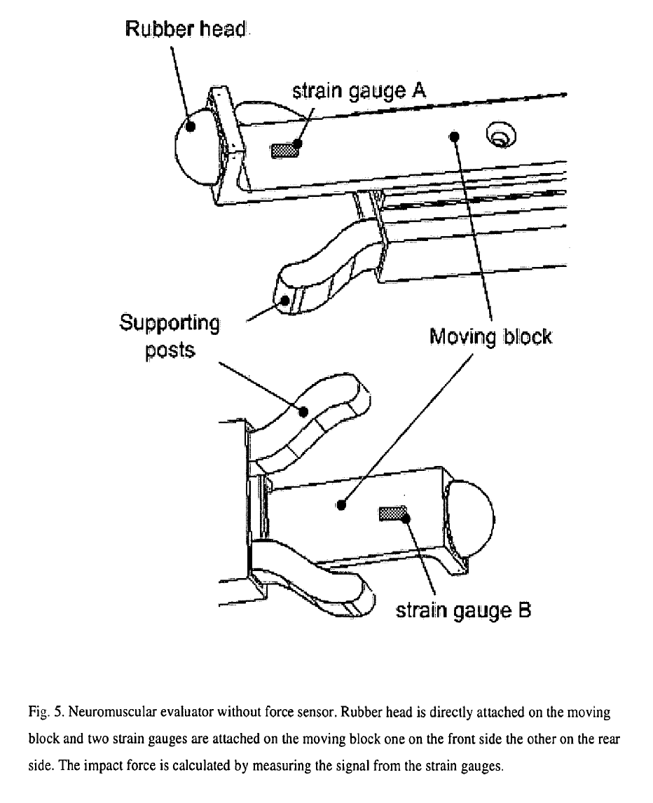

There are several alternative designs for mounting the force sensor. In the first design, the force sensor is mounted on the moving block and the force sensor is covered by the rubber head. Force sensor and rubber head move together with the moving block (FIG. 4a). In the second design, the force sensor with the rubber head mounted on is separated from the moving block (FIG. 4b). There is an elastic strap connecting the two supporting posts. The force sensor with the rubber head affixed at the center of the strap so that the sensor contacts with the tendon when the supporting posts are placed at a proper position. The moving block hits the force sensor which is always in contact with the tendon, and the impact force is transferred to the tendon. The moving block retracts back when the desired force threshold is reached but the force sensor still remains in contact with the tendon which measures the reflex-mediated tendon bounce-back force as well as the tapping force. In the third design, strain gauges can be mounted on the moving block to measure the tapping force, similar to the first design but combining the force sensor with the moving block (FIG. 5).

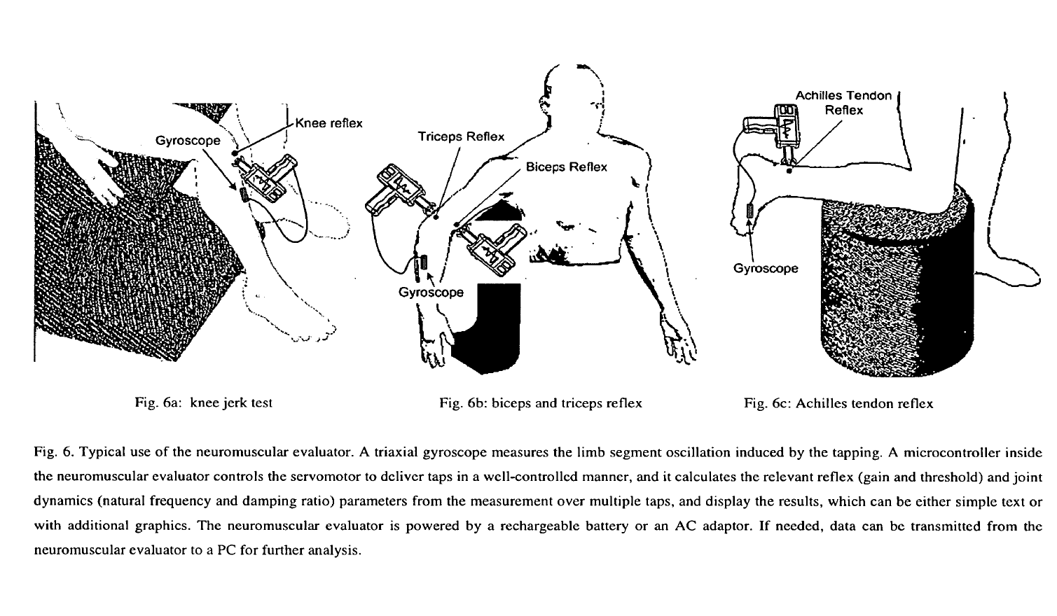

Limb oscillation can be measured using a gyroscope (FIG. 6). The gyroscopic sensor measures the rotation rate instead of the joint angle directly. An advantage of that over direct angle measurement is that it offers immunity to shock and vibration. Furthermore, the gyro sensor has high dynamic range and a small footprint which can be easily packaged into a small enclosure. It is attached to the anterior leg through an adaptor to match the limb surface using double-sided tape or a strap with wire connection to the neuromuscular evaluator to measure the knee flexion/extension oscillation induced by the patellar tendon tapping (FIG. 6a). If triaxial gyroscope is used to measure the angular rates in three axes, tri-axial tilt angles can be measured by integrating the angular rates. A miniature tri-axial accelerometer or an inclinometer can also be utilized to measure the limb oscillation as alternative options. The small gyroscope (or inclinometer or accelerometer) can be wired to the main evaluator body or a wireless communication module can be used for a wireless transmission.

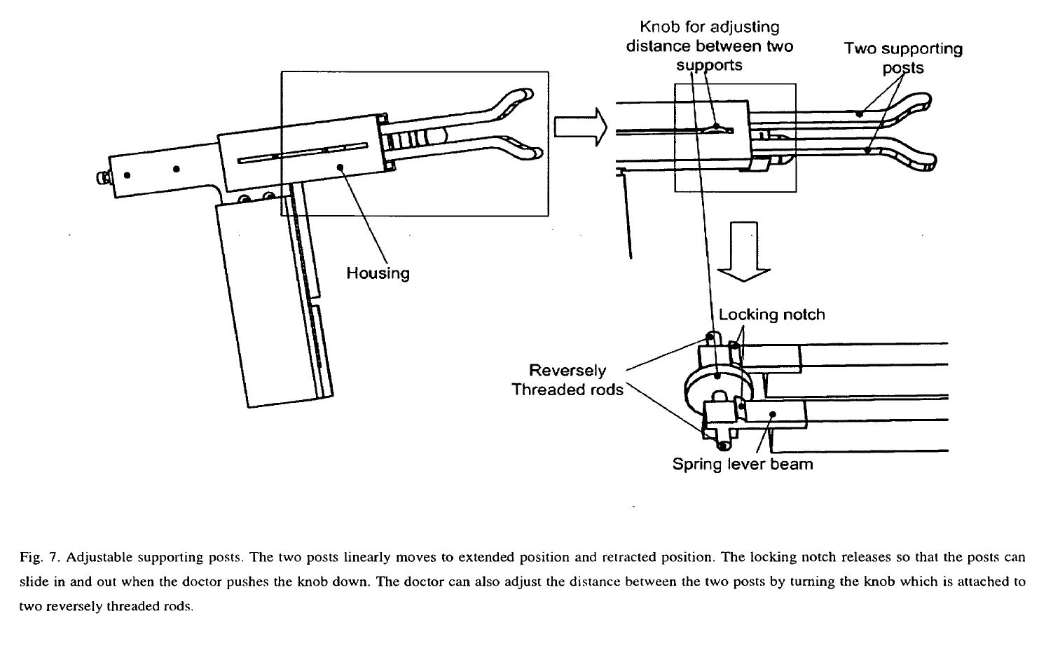

The neuromuscular evaluator is positioned and supported during the tapping using a pair of supporting posts on the front end of the neuromuscular evaluator, which stabilized the device and insure consistent impact location and strength. The supporting posts are designed so that the posts can be extended out when the meter is in use or they can be retracted for convenient storage when the neuromuscular evaluator is not in use. The interval between the two posts is adjusted by turning a knob to which two threaded rods with reversed threads are attached (like the width adjustment between two legs of a regular compass) (FIG. 7). The interval is adjusted to be slightly wider than the tendon width. The clinician holds the neuromuscular evaluator steadily against the limb so that it is stable during the tapping, which is important in obtaining reliable tapping control and reflex measurements.

The clinician may adjust the neuromuscular evaluator positioning to find the most sensitive spot on the tendon to elicit reflexes, similar to what is done in a clinical examination using a traditional reflex hammer. With the neuromuscular evaluator, the tapping force can be adjusted conveniently by turning the force threshold adjustor knob (FIG. 1). Each time the ‘Trigger button’ is pressed, the neuromuscular evaluator accelerates the moving block quickly to hit the tendon with strong impact until the tapping force reaches a target level. The moving block is then retracted quickly and returned to the initial position, resulting in a brisk tap onto the tendon and getting ready for the next tap. The peak tapping force may be adjusted automatically to elicit clear but moderate reflex responses and thus determine the threshold in tapping force. A few (˜3) taps is then delivered at the level slightly above the threshold for the neural and muscular evaluations.

The microcontroller calculates the tendon reflex gain (from the tapping force input to the knee oscillation output measured by the gyro) and the reflex threshold in tapping force, natural undamped frequency and damping ratio of the knee joint dynamics, display the results on a LCD or OLED immediately after the taps, and save it in the device if needed. The clinician can control/adjust the neuromuscular evaluator for individual subjects and specific tendons and get the feedback from the display (FIG. 1).

### 2. Control of the Neuromuscular Evaluator

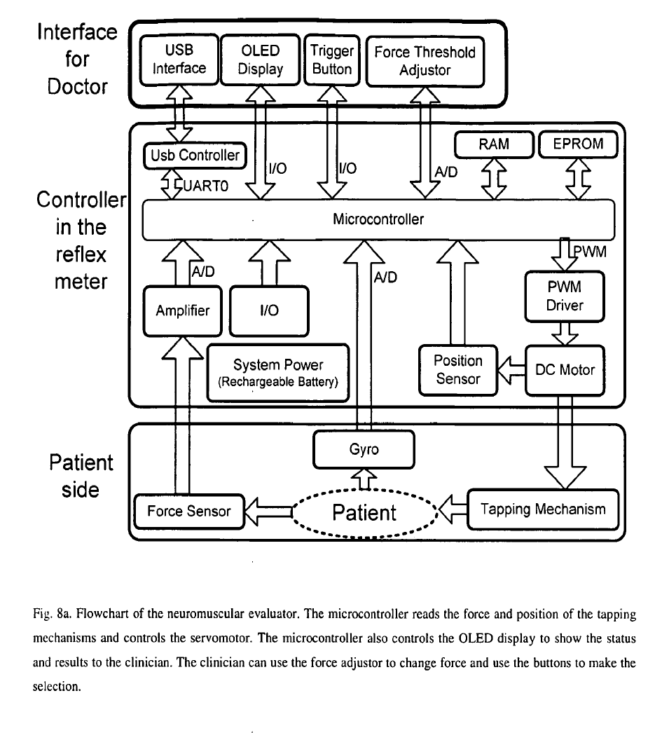

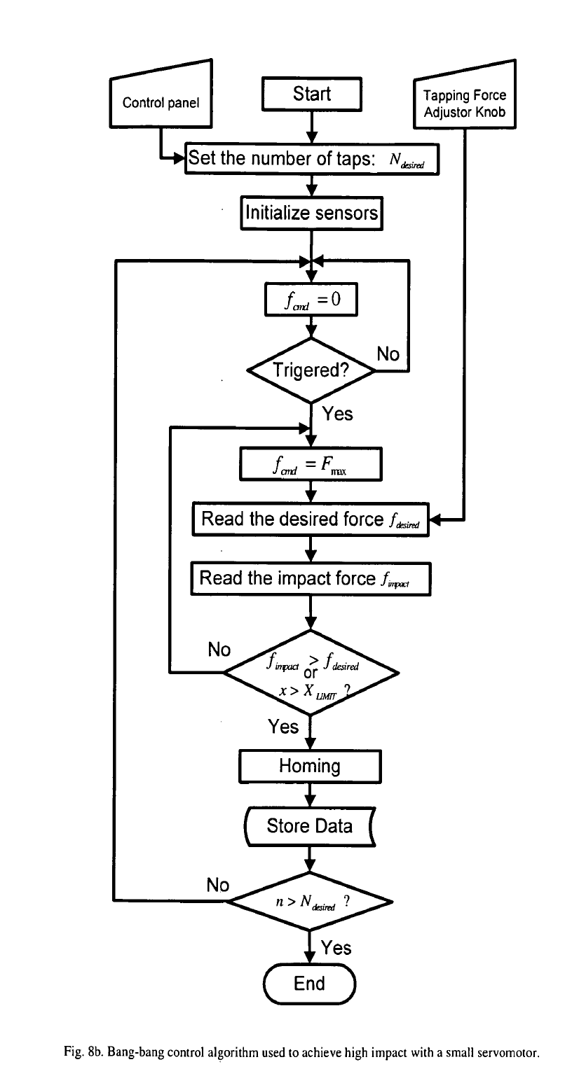

The tendon taps is delivered by a servomotor and controlled by a microcontroller (FIG. 8). Since different subjects may need quite different levels of tapping force and the threshold in tapping force is an important measure of reflex excitability, the neuromuscular evaluator taps over a common range of force levels to determine the reflex threshold. The clinician may also select custom range of tapping force through the force threshold adjustor (FIG. 1). The microcontroller reads the position and force signals and controls the servomotor accordingly (FIG. 8a)

The impact to the tendon is related to the velocity and mass of the moving block, and it needs to be brisk for proper reflex activation. In order to generate strong enough impact to elicit reflex using a small motor, the moving block needs to be accelerated to reach the maximum velocity quickly and the impact contact time Δt should be small for a brisk tap. First, a bang-bang impact force control is implemented for the purpose (Slotine, 1991). The bang-bang controller accelerates the moving block as much as possible using a maximal force command (f_{cmd}=F in FIG. 8b). Second, as soon as the impact force reaches a desired level (f_{d}), the controller retracts the moving block quickly and return it back to the initial position (‘homing’ in FIG. 8b), resulting in a brisk tap onto the tendon (FIG. 8b). Third, a relatively long rail is used so that the moving block can be accelerated to the maximum velocity before the impact. Several taps are delivered consecutively with the peak tapping force slightly above the threshold and the several reflex and joint dynamics measures are determined over the multiple taps.

### 3. Procedure

The neuromuscular evaluator is tested on human subjects to evaluate its performance. For the patellar tendon reflex, the subject sits on a seat with the leg suspended (FIG. 6a). The subject is asked to relax and not to react to the taps. Tendon tapping force and knee joint movement are measured by the force sensor and inclinometer, respectively (FIG. 6a). If needed, EMG signal of the involved muscle may be measured in some cases as further corroboration. The user holds the pocket neuromuscular evaluator against the knee with the two supporting posts resting on the medial and lateral sides of the patellar tendon (FIG. 6a). Once neuromuscular evaluator is in place and the most sensitive spot is located, the user then starts the sequence of several taps around the threshold level to determine the several reflex and joint dynamics parameters.

Similar tests can be done at the triceps (FIG. 6b) and biceps (FIG. 6b) tendons at the elbow and the Achilles tendon at the ankle (FIG. 6c). The upper arm (or leg) is supported and/or held in place by the clinician during the test, while the forearm (or foot) is free to swing as the tapping-induced responses. Both reflex excitability (reflex gain and threshold) and joint mechanical properties (natural frequency and damping ratio) are evaluated.

The neuromuscular evaluator can also be used for evaluating non-reflex properties such as the passive/active joint ROM (range of motion), active muscle strength, and joint stiffness. Passive and active joint ROM is measured by the gyro/accelerometer. The clinician grasps and moves patient's limb within the ROM while the gyro/accelerometer measures joint angles to evaluate passive ROM. Integration may be used to obtain joint angle from the angular rate/acceleration measurement. The tilt measurement provided by an inclinometer may also be used for the angular ROM measurement. Active ROM is measure in a similar way but the patient actively moves the limb while the gyro/accelerometer measures the joint angles. To measure the muscle strength, the clinician holds the neuromuscular evaluator with its supporting posts and moving block retracted to initial position. The patient is asked to push against the rubber bump while the doctor resists against the patients by holding the evaluator. The force sensor in the evaluator measures the force generated by the patient for determination of the muscle strength. In the similar way, the clinician can exert force to patient's limb through the evaluator while the neuromuscular evaluator measures the resistance force and joint movement simultaneously to determine joint stiffness.

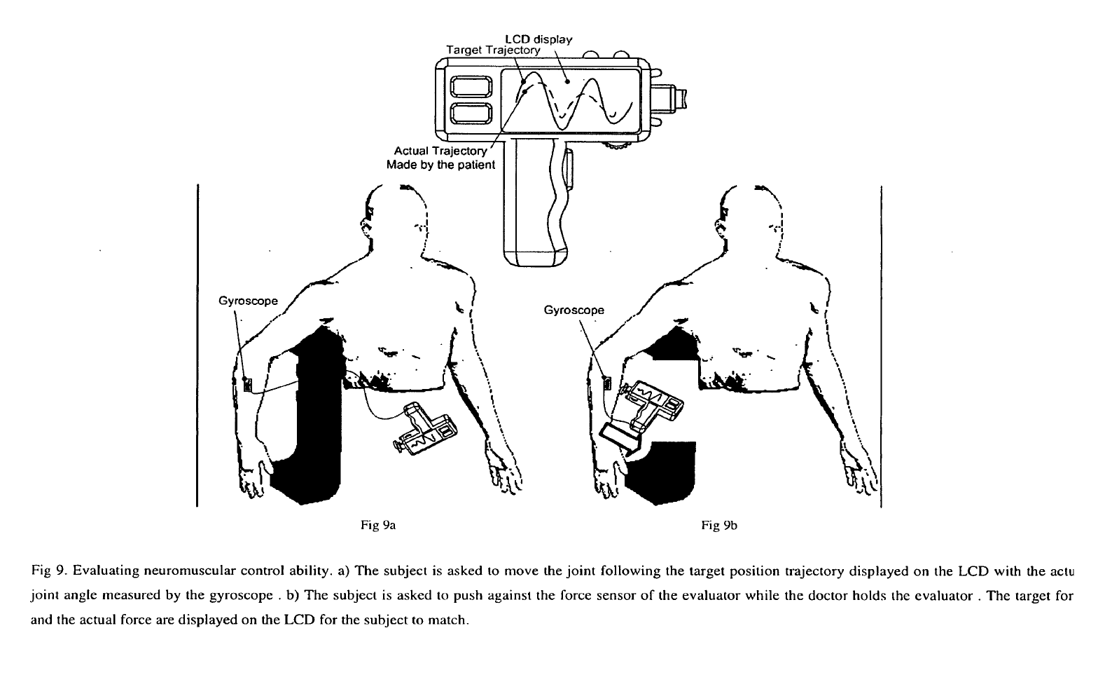

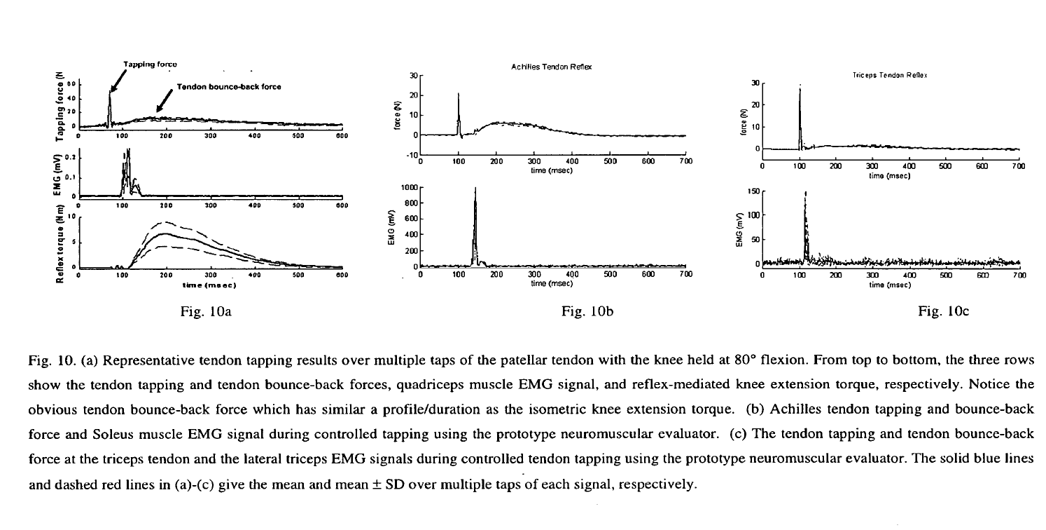

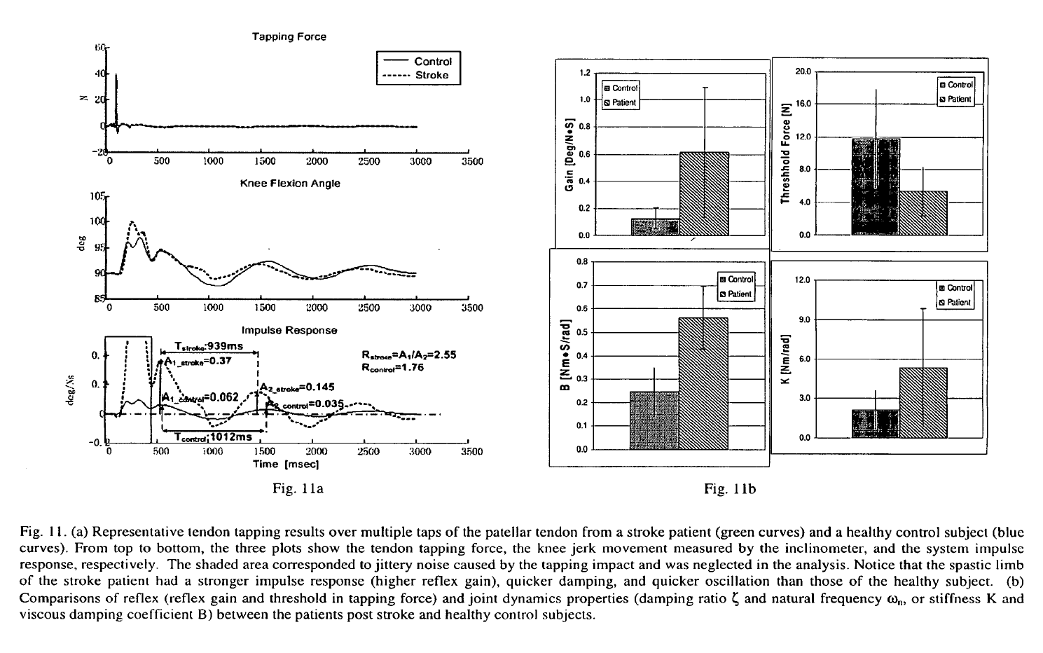

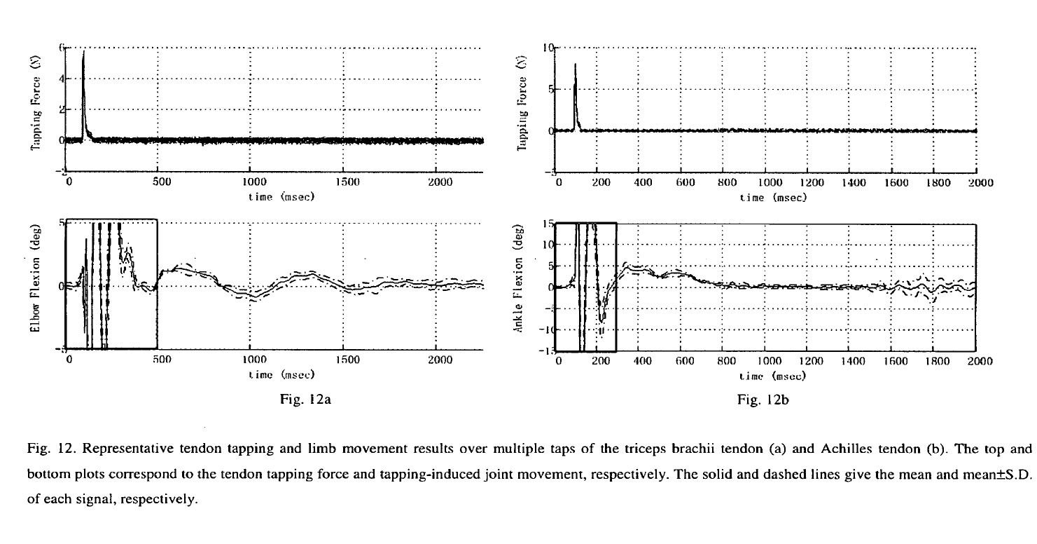

The neuromuscular evaluator can also be used for evaluating the patient's neuromuscular control ability. First, it can evaluate the patient's ability to control position of a joint. The patient is asked to move his/her limb to follow the target joint position trajectory displayed on the LCD. The target trajectory and the actual joint angle are displayed simultaneously on the LCD so that the patient can adjust his/her movement to reduce the error between the two curves (FIG. 9a). Second, the neuromuscular evaluator can also be used to assess the patient's ability to control force (FIG. 9b). The patient is asked to push against resistance as the clinician holds the evaluator against the patient's limb. The target force and the actual force generated by the patient are displayed simultaneously. The central processing unit then calculates the patient's ability to control the voluntary movement/force by analyzing the data.

### 4. Determination of the Tendon Reflex System Measures

The impulse response is used to characterize the tendon reflex system with the reflex-mediated limb movement as the system output and the tapping force as the input, respectively. Since the tapping force is very brief, it can be approximated as a pulse and the impulse response h_{fθ}(t) can be conveniently approximated as the reflex-mediated limb movement response θ(t) scaled by the area of the tapping force pulse fₜ(t):

$${h}_{f θ} ( t ) = \frac{θ ( t )}{{\sum }_{τ} {f}_{t} ( τ )} \qquad ( 1 )$$

The simple scaling method resulted in multiple impulse responses, one for each tap. Statistics can be done over the taps to get more reliable results. The area of the impulse response h_{fθ}(t) can be used as the gain of the tendon reflex system, the rising slope of h_{fθ}(t) as the contraction rate, and the delay from the tapping force peak to the onset of h_{fθ}(t) as the reflex-loop delay. Practically, it is easier to use the peak of the impulse response h_{fθ}(t) as the tendon reflex gain. The reflex threshold in tapping force can be characterized as the level of peak tapping force beyond which reflex responses are induced. So the tendon reflex gain and reflex threshold in tapping force that can be determined conveniently are included for the clinical uses.

### 5. Analysis of Spastic Joint Dynamics Using Pendular Motion Induced in Tendon Reflexes

The limb pendular motion induced by tendon tapping can be described by the following lumped model and it is used to characterize the biomechanical changes in spastic muscle-joints (Lin and Rymer, 1991).

I [symbol] (t)+B\dot{θ}(t)+Kθ(t)+mgl_{c} sin θ(t)=0 (2)

where θ(t) is the joint angle as a function time t. I, B and K are the limb inertia, joint viscosity and joint stiffness, respectively. m, l_{c}, and g are the limb mass, the distance from the limb center of mass to the joint rotation axis and acceleration due to gravity, respectively. For small amplitude rotations about the limb vertical position, sin(θ)≈θ and the above equation is approximated as

I [symbol] (t)+B+\dot{θ}(t)+(K+mgl_{c})θ(t)=I [symbol] (t)+B\dot{θ}+K′θ(t)=0 (3)

where K′=K+mgl_{c}. The above equation can also be represented by the natural (undamped) frequency (ωₙ) and damping ratio (ζ) as follows:

I [symbol] (t)+2ζωₙ\dot{θ}(t)+ωₙ²θ(t)=0 (4)

Considering that human joints are generally under-damped system with 0<ζ<1 (Agarwal and Gottlieb J Biomech Eng 99: 166-170, 1977; Zhang et al. J Biomech 31: 71-76, 1998; Zhang et al. J Orthop Res 18: 94-100, 2000) the pendular oscillations can be described in the form of θ(t)=K′e^{-αt} cos ωt. From either the impulse response of the tendon reflex or the pendular motion itself, we can measure the ratio of the peak angle of one cycle to the peak angle of the next cycle (R) and the period of a cycle (T). Although 3 unknowns are involved in Eqs. (2), (3) or (4), the damping ratio ζ and natural frequency ωₙ can be determined from the above 2 measures as follows:

$$ζ = \sqrt{\frac{{( ln R )}^{2}}{4 {π}^{2} + {( ln R )}^{2}}} and {ω}_{n} = \frac{2 π}{T \sqrt{1 - {ζ}^{2}}} \qquad ( 5 )$$

Variables in Eqs. (3) and (4) are related to each other (Kearney and Hunter, CRC Crit Rev Biomed Eng 18: 55-87, 1990; Zhang et al. J Biomech 31: 71-76, 1998; Zhang et al. J Orthop Res 18: 94-100, 2000) and the equations below show one way of the transformations. If needed, the moment of inertia of the limb, I, can be calculated from the anthropometric data of the limb (Winter, Biomech Motor Control Human Movement, 2000)

$$ζ = \frac{B}{2 \sqrt{{IK}^{′}}} and {ω}_{n} = \sqrt{\frac{{K}^{′}}{I}} \qquad ( 6 )$$

Practically, the measures of the damping ratio ζ and natural frequency ωₙ can be used as the measures of limb dynamic properties provided by the neuromuscular evaluator since they cover the main characteristics of the limb dynamics.

### 6. Characterization of Neuromuscular Changes Associated with Spasticity

Changes in neuromuscular properties associated with spasticity are evaluated at the patellar tendon in stroke patients with leg/arm spasticity and healthy controls using the prototype neuromuscular evaluator. The subject is seated comfortably with the leg freely suspended, the reflex hammer is used to tap the patellar tendon and record the tapping force, while the knee jerk movement is measured by an inclinometer as the reflex response (FIG. 1b). Tendon reflexes are analyzed through system identification with the tendon tapping force as system input and reflex-mediated knee movement as the output. Specifically, the reflex excitability is characterized by the reflex gain and reflex threshold in tapping force, and the knee joint dynamics are characterized by the natural frequency and damping ratio. In summary, the neuromuscular evaluator characterize significant changes in the neuromuscular reflex properties associated with spasticity, including increase in reflex excitability (higher reflex gain and lower threshold in tapping force) and increased damping (higher ζ) and higher natural frequency ωₙ, or in alternative representations, higher stiffness K and higher viscous damping B (FIG. 11).

Similar tendon reflex measurements are done at other tendons, including the triceps (and biceps) tendon at the elbow (FIG. 12a) and Achilles tendon at the ankle (FIG. 12b). With the taps consistently delivered at the most sensitive spot by the well-controlled neuromuscular evaluator, the reflex responses are repeatable, which provides a potentially reliable way to quantify tendon reflexes consistently.

What is claimed is:

1. A neuromuscular evaluator capable of eliciting tendon reflexes by tapping onto a tendon under precise control.

2. The design of 1 wherein an actuator, said actuator generating rotary or linear motion such that the motion can be used to tap onto a tendon. Electric motor (FIG. 2) or mechanical spring (FIG. 3) can be used as the actuator.

3. The design of 1 wherein a cable-driven mechanism is used, the said cable-driven mechanism converts the rotary motion to linear motion in case a rotary motor is used.

4. The design of 1 wherein a rack-and-pinion mechanism is used, the said rack-and-pinion mechanism converts the rotary motion to linear motion as an alternative for the cable-driven mechanism.

5. The design of 1 wherein a force sensor, said force sensor mounted on one end of the moving block.

6. The design of 1 wherein a rubber pad, said rubber pad mounted in front of the force sensor.

7. The design of 1 wherein a central processing unit, said central processing unit controlling the current flows to the motor based on the force measured at the said force sensor. The central processing unit is capable of analyzing and saving collected data.

8. The design of 1 wherein a display unit, said display unit displaying the collected and analyzed data.
A LED, said LED displaying the mode of operation by different colors (e.g., Green color—ready)
A control panel, said control panel that has buttons to move cursors on the screen or to input commands.

9. The design of 1 wherein a rechargeable battery, said rechargeable battery storing electric power to run the apparatus for a couple of hours.

10. The design of 1 wherein a trigger button, said trigger button sending electric signal to the central processing unit to initiate the motion at the motor.

11. The design of 1 wherein a force threshold adjustor, said force threshold adjustor setting the desired value of impact force so that the central processing unit can control to retract the moving block when the desired impact force is reached.

12. The design of 1 wherein a gyroscope, said gyroscope measuring the angular rotation rate.

13. The design of 1 wherein a triaxial accelerometer, said accelerometer measuring the angular acceleration and tilt angle.

14. The design of cable mechanism in 3 wherein the cable-driven mechanism comprising:
A small tube, said tube affixed to motor shaft and two cables are affixed to the tube.
A moving block, said moving block mounted on a said linear motion guide. The moving block moves along the direction of the linear motion guide.
Two cables, said two cables connecting the said moving block with the said tube. One end of a cable wraps around the said tube and the other end of the cable is fixed to one end of moving block. One end of the second cable wraps around the tube in the opposite direction and the other end of the second cable is affixed to a cable tensioner.
A cable tensioner, said cable tensioner linearly pulling the cable by turning a said screw. One end of the said cable is affixed to the said tensioning block where the said screw is mounted on. As the screw is turned, the tensioning block moves linearly and pulls the cable into tension.

15. The mechanism for supporting post(s) in apparatus of 1 comprising:
Supporting post(s), said supporting post(s) that stand on the limb for stable and reliable positioning of neuromuscular evaluator and the tapping impact location,
A knob, said width adjusting knob allowing the adjustment of the distance between the two supporting posts by turning the knob.
A spring lever beam, said spring lever beam allows elastically pushing the knob down to release the lock.
A housing, said housing covering the two supporting posts and allowing linearly sliding the posts in and out.

16. The method to diagnose/evaluate neurological reflex and muscle joint biomechanical properties of the subject being tested.

17. The method of 16 wherein the two supporting posts are extended and the distance between two posts are adjusted to be slightly larger than the width of tendon.

18. The method of 16 wherein the clinician sets the threshold value of the impact force by turning the force threshold adjustor.

19. The method of 16 wherein the clinician attaches the gyro onto the limb that is to move during the test.

20. The method of 16 wherein the clinician clicks triggering button to initiate the tap.

21. The method of 16 wherein the central processing unit controls current flowing into the motor to reach a high speed at the impact to the tendon.

22. The method of 16 wherein the moving block linearly travels toward the target point at a high speed as the motor rotates with its high speed.

23. The method of 16 wherein the rubber pad taps/impacts the tendon as the moving block travels toward the target point.

24. The method of 16 wherein the central processing unit reads the force data measured at the force sensor.

25. The method of 16 wherein the central processing unit sends control commands to the motor to rotate the motor in the opposite direction when the desired force threshold values is read from the force sensor.

26. The method of 16 wherein the central processing unit sends out control commands to stop the motor rotation as the moving block reaches the initial (retracted) position.

27. The method of 16 wherein the central processing unit reads the limb oscillation measured by the gyro or accelerometer and displays the result on the screen.

28. The method of 16 wherein the central processing unit calculates the reflex gain and threshold in tapping force parameters based on Equations (1) and displays the results on the screen.

29. The method of 16 wherein the central processing unit calculates natural undamped frequency and damping ratio of muscle joint system, and stiffness and viscous damping parameters by using equations (5) and (6) and displays the results on the screen.

30. The method of 16 wherein the central processing unit waits for the next trigger signal to execute next tendon reflex tap.

31. The method of 16 wherein the doctor retracts the supporting posts when the test is completed.

32. The method of 16 wherein the clinician places the neuromuscular evaluator at various tendon locations such as the patellar, triceps brachii, biceps brachii, and Achilles tendons.

33. The method to diagnose muscle joint biomechanical properties of the muscles joint involved.

34. The method of 33 wherein the gyro (or accelerometer) measures rotation rate (or acceleration) and the central processing unit integrates the measured values to determine the joint angle.

35. The method of 33 wherein the gyro (or accelerometer) measures joint rotation to evaluate active joint range of motion while the patient moves his/her limb to the joint limits.

36. The method of 33 wherein the gyro (or accelerometer) measures joint rotation to evaluate passive joint range of motion while the clinician moves the subject's limb to the joint limits.

37. The method of 33 wherein the clinician measures active muscle strength of the subject.

38. The method of 37 wherein the clinician holds the evaluator with the rubber pad placed on the subject's limb.

39. The method of 37 wherein the clinician asks the subject to actively push against the rubber pad while the doctor resists against the patient's movement.

40. The method of 37 wherein the force sensor measures the force value and the central processing unit calculates joint strength by multiplying the maximum force by the moment arm which is the linear distance from the joint to the location of rubber pad.

41. The method of 33 wherein the evaluator measures joint stiffness.

42. The method of 41 wherein the doctor holds the evaluator with the rubber pad contacting with the patient's limb.

43. The method of 41 wherein the doctor moves the limb through the evaluator to the joint limit while the force sensor measures resisting force and the gyro (or accelerometer) measures the joint angles.

44. The method of 41 wherein the central processing unit calculates the joint torque by multiplying the force by the moment arm which is the linear distance from the joint to the rubber bump.

45. The method of 41 wherein the central processing unit calculates joint stiffness by dividing the change in joint torque by the corresponding change in joint angle.

46. The method of 41 wherein the evaluator displays the values of passive joint ROM, active joint ROM, active muscle strength, and joint stiffness.

47. The method of 33 wherein the evaluator measures neuromuscular control ability of a patient

48. The method of 47 wherein the evaluator displays target trajectory on the LCD panel while the joint angle measured from the gyroscope (or accelerometer) is displayed simultaneously on the LCD.

49. The method of 47 wherein a patient is asked to follow the target joint position trajectory displayed on the LCD by moving his/her joint and matching the actual joint angle with the target trajectory.

50. The method of 47 wherein a patient is asked to follow the target trajectory displayed on the LCD by pushing against the clinician's resistance and matching the actual torque generated with the target torque trajectory.

51. The method of 47 wherein the central processing unit calculates the neuromuscular control ability of a patient by comparing the target position/torque trajectory with the actual position/torque curve the patient generated.
